(19) United States
(12) Patent Application Publication
(10) Pub. No.: US 2007/0192136 A1
(43) Pub. Date: Aug. 16, 2007

(54) SYSTEMS AND METHODS FOR FACILITATING MEDICAL ORDER FULFILLMENT

(75) Inventors: Randolph B. Lipscher, Austin, TX (US); Eric Wohl, Austin, TX (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: CATALIS, INC, Austin, TX (US)

(21) Appl. No.: 11/668,164

(22) Filed: Jan. 29, 2007

Related U.S. Application Data
(60) Provisional application No. 60/763,124, filed on Jan. 27, 2006.

(51) Int. Cl. G06Q 10/00; A61B 5/00

(52) U.S. Cl. 705/2; 600/300

(57) ABSTRACT

A method of establishing an order includes receiving discrete medical findings data associated with a patient, receiving order data associated with the patient, receiving findings selection data indicative of at least a portion of the discrete medical findings data, and communicating the order data and select findings data to a separate service provider. The select findings data include at least the portion of the discrete medical findings data. In addition, a computer readable media may include computer-implemented instructions for performing such a method.

### CORRESPONDING APPLICATIONS

The present application claims priority from U.S. Provisional Patent Application No. 60/763,124, filed Jan. 27, 2006, entitled “SYSTEMS AND METHODS FOR FACILITATING MEDICAL ORDER FULFILLMENT,” naming applicants Randolph B. Lipscher and Eric Wohl, which application is incorporated by reference herein in its entirety.

### TECHNICAL FIELD OF THE DISCLOSURE

This disclosure, in general, relates to systems and methods for facilitating medical order fulfillment.

### BACKGROUND

Increasingly, medical practices are turning to computational systems for managing patient data, appointment scheduling, and financial functions. In particular, hospitals rely on large integrated computer systems for tracking patients and supplies and for managing financial transactions, such as third party payer billing. Although such large integrated systems are improving with technology, such systems generally suffer from poor interfaces with healthcare providers and large overhead costs associated with management of computational systems. Typically, the large overhead cost associated with large integrated hospital computer systems makes such systems impractical for use in ambulatory medical facilities.

Ambulatory medical practices are also turning to computational systems for storing patient medical data, for scheduling appoints, and for tracking payment for services. Frequently, ambulatory medical practices, such as general physicians offices, or orthopedic specialists located outside of a hospital environment, rely on separate external service providers for facilitating or fulfilling medical orders, such as radiological orders or laboratory testing. In contrast to the large integrated hospital systems, computer systems typically available to ambulatory medical practices do not provide integration with computer systems of separate and remote service providers.

Moreover, recent changes to privacy laws within the United States restrict sharing of patient medical data between entities. Such restrictions apply to both large integrated hospital systems and separate ambulatory medical practices. In general, computational systems suitable for use in hospital environments or those computational systems suitable for use in ambulatory medical practices either fail to permit sharing of relevant patient data in accordance with privacy laws or permit an excessive amount of sharing, which may violate such laws.

As such, an improved computational system for use in ambulatory medical practices would be desirable.

### BRIEF DESCRIPTION OF THE FIGURES

The present disclosure may be better understood, and its numerous features and advantages made apparent to those skilled in the art by referencing the accompanying drawings.

FIG. 1 includes an illustration of an exemplary computational system for facilitating order fulfillment.

FIG. 2 includes an illustration of an exemplary interface to collect discrete medical findings.

FIGS. 3, 4, 5, 6, and 7 include illustrations of exemplary methods for facilitating order fulfillment.

FIGS. 8, 9, 10, 11, 12, 13, and 14 include illustrations of exemplary interfaces for use in computational systems, such as the computational system illustrated in FIG. 1.

### DETAILED DESCRIPTION

In a particular embodiment, a system includes an encounter management system and a service provider system in communication with the encounter management system. The encounter management system is located at an ambulatory medical facility and the service provider system is located at a remote and separate ambulatory facility. In addition, the system may include a consultant system and an accounting system in communication with the service provider system. In a particular example, the encounter management system is configured to receive discrete medical findings data associated with a patient and to receive order data associated with that patient. The order data may be indicative of a medical order, such as an order for a medical procedure, imaging, or test. The order data may, for example, include an identifier of the order, an identifier of the encounter management system, and specific data about the order, such as which tests are to be performed, which methodologies are to be followed, and contact information associated with the patient. In addition, the encounter management system may receive finding selection data and communicate the order data and select findings data indicated by the finding selection data to a separate service provider system. Further, the service provider system may receive the order data and the select findings data and facilitate collection of order results data. The order results data and the select findings data may be communicated to a consultant system and the consultant system may provide consultant input data to the service provider system. The service provider system may forward the order results data and the consultant input data to the encounter management system. In response, the encounter management system may provide an interface to a healthcare professional based at least in part on the order results data and the consultant input data. The encounter management system and the service provider system may communication order status with the accounting system.

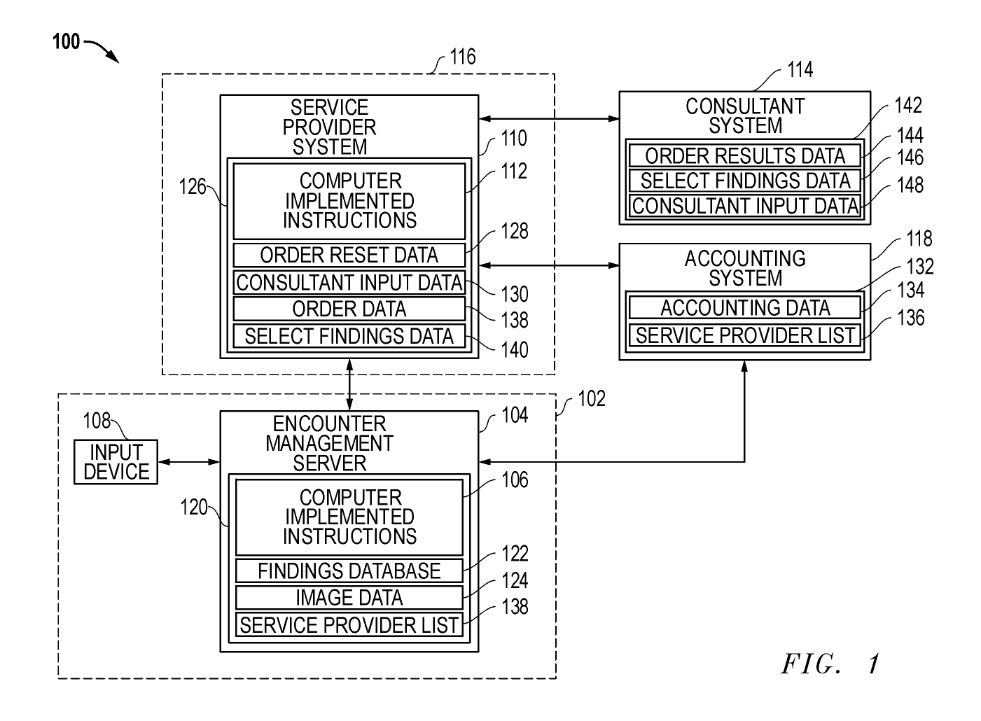

As illustrated in FIG. 1, a system 100 may include an encounter management system 104 in communication with a service provider system 110. The encounter management system 104 is located at an ambulatory medical facility 102 separate and distinct from the location 116 of the service provider system 110.

Within the ambulatory medical facility 102, the encounter management system 104 may communicate with an input device 108. For example, the encounter management system 104 may provide interfaces and receive discrete medical findings data, finding selection data, and order data from the input device 108. In general, a finding is a single data element indicating a condition, a diagnosis, a complaint, a quality associated with a condition, a pathology, or any combination thereof In particular, a discrete medical finding includes an indicia or a medical finding such as a code, word, or tag of a medical vocabulary indicative of the medical finding. In an exemplary embodiment, the discrete medical finding is associated with a symptom. In another example, discrete medical finding is associated with a chief complaint. In a further example, the discrete medical finding is associated with a physical exam. In addition, the discrete medical finding may be associated with a diagnosis.

Order data is associated with an order requested by a healthcare professional. The order may be a request for a test, imaging, or procedure. In a particular example, the order is to be performed at a separate facility from the clinic at which the order is requested. The order data may, for example, include an order identifier, an ordering clinic identifier, and specific information about the order, such as which tests are to be performed, which methodologies are to be followed, and contact information for the patient. In addition, the order data may include a fulfillment clinic identifier. In a particular embodiment, the order data may also include patient scheduling information. For example, a laboratory order may include an order identifier, such as an identification number, patient information, such as a contact number or insurance information, and specific data associated with the order, such as, for an exemplary blood test, a request for performing a blood glucose level, an HIAC test, a total cholesterol test, and a triglyceride test. For an exemplary radiological order, the order data may include an order identifier, patient contact information, the type of radiological test, such as X-ray, CAT scan, or MRI, and an anatomical location. In a particular embodiment, radiological order data may include an anatomical drawing of a body or a portion of the body with an indicator as to locate the location of which the healthcare provider desires imaging. In a particular example, the input device 108 is a wireless computational device, such as an ultraportable computer or pad device, which interacts with a healthcare professional, such as a physician, nurse, or medical practitioner.

The encounter management system 104 includes computer-implemented instructions 106 and a networking interface to the input device 108, such as a wireless or a wired networking interface. The computer-implemented instructions 106 may be stored in a memory 120 and may be operable by a processor within the encounter management system 104 to implement methods and functionality associated with the encounter management system 104. For example, the computer implemented instructions 106 may be operable by a processor to provide a discrete medical findings interface to the input device 108 and to receive data entered in the discrete medical findings input interface from the input device 108. In addition, the encounter management system 104 may provide an order interface to the input device 108 and may receive order data indicative of an order from the input device 108. Further, the encounter management system 104 may provide a findings selection interface to the input device 108. Through such an interface, a healthcare professional may select findings data to attach to an order. In addition, the encounter management system 104 may provide a service provider selection interface to the input device 108 through which a healthcare professional may select a service provider to whom the encounter management system 104 may forward the order data and selected findings data. In an exemplary embodiment, a list of service providers 136 is provided to the encounter management system 104 from the accounting system 118 as requested. Alternatively, the encounter management system 104 may periodically download the service provider list and store the service provider list in memory 120.

In an exemplary embodiment, the encounter management system 104 forwards order data and select findings data based on the findings selected by a healthcare professional to a service provider system 110. For example, the encounter management system 104 may communicate with the service provider system 110 through a dial-up modem or a network, such as a wide-area network or a global Internet. In addition, the service provider system 110 may communicate with the consultant system 114 and an accounting system 118 via a dial-up modem or a network.

The service provider system 110 may include computer-implemented instructions 112 stored in a memory 126 and operable by a processor to facilitate order data management and communication with systems, such as the encounter management system 104, the consultant system 114, and the accounting system 118. Upon receiving the order data and selected findings data, the service provider system 110 may store the order data 138 and select findings data 140 in the memory 126 and may initiate collection of data associated with the filling an order indicated by the order data 138. For example, the order may be a medical order relating to lab tests or radiological imaging to be performed on a patient. Initiating an order may, for example, include contacting the patient to schedule an appointment or may include collecting samples. In a particular example, the order relates to a radiological test such as a magnetic resonance imaging (MRI) image, an X-ray image, or a CAT scan image. In such an example, initiating an order may include scheduling time with a patient and performing the radiological test on the patient. Once the test is complete, the service provider system 110 may receive and store order results data 128 associated with the order. The order results data may, for example, include an order identifier, test results data, image data, or any combination thereof. In a particular example, the order results data may also include an identifier associated with the requesting clinic 102 or encounter management system 104

Upon receiving the order results data 128, the service provider system 110 may communicate with the consultant system 114. For example, the consultant system 114 may interface with a healthcare professional who provides input on test results, such as a radiologist. In addition, the service provider system 110 may provide the select findings data 140 received from the encounter management system 104 to the consultant system 114. The consultant system 114 may be located at the remote ambulatory facility 116 or may be located in a separate facility. Alternatively, the consultant system 114 may be implemented as a portion of or an interface to the service provider system 110.

In an exemplary embodiment, a healthcare professional, such as a radiologist, provides consultant input data 148 for storage in a memory 142 of the consultant system 114. Consultant input data 146 may include an order identifier and test findings data. Test findings data may, for example, include text or indicators of discrete medical findings. The consultant system 114 forwards the consultant input data 148 to the service provider system 110. The service provider system 110 may store the consultant input data 130 and may associate the consultant input data 130 with the order results data 128 and forward the order results data 128 and consultant input data 130 to the encounter management system 104. The consultant system 114 may also store the order results data 144 and select findings data 146 in memory 142.

After receiving the order results data 128 and before forwarding the order results data 128 to the encounter management system 104, the service provider system 110 may convert the data into a format for easy communication. Generally, in exemplary radiological applications, the order results data may be in a DCIM format. In a particular embodiment, the service provider system 110 may convert the DCIM format image to a compressed Internet format, such as JPEG, GIF, PNG, Bitmap, MPEG, or any combination thereof

In response to completion of the order, including taking or receiving the order results data 128, and optionally receiving the consultant input data 130, the service provider system 110 may communicate accounting information, such as order status, to the accounting system 118. The accounting system 118 is generally located at a separate facility from the ambulatory facilities 102 and 116. Alternatively, the accounting system 118 may be located at one or both of the facilities 102 and 116.

In response to receiving the order results data and optionally, the consultant input data, the encounter management system 104 may store the order results data and optionally, the consultant input data in a storage memory, such as a database. In a particular example, the encounter management system 104 stores the consultant input data in a discrete medical findings database 122. In general, the discrete medical findings database 122 is configured to store discrete medical findings. In another example, the encounter management system 104 stores the order results data, such as an image, in an image format 124 and includes entries within a discrete medical findings database 122 to indicate the presence of such order results data. In addition, the encounter management system 104 may provide an order results interface to the input device 108 based at least in part on the order results data and optionally, the consultant input data.

In response to completion of the order, the encounter management system 104 may communicate with the accounting system 118 to indicate receipt of the order results data. The encounter management system 104 may also communicate with the accounting system 118 to indicate the existence or status of an order when the order data is received from the input device 108.

The accounting system 118 receives input from the encounter management system 104 and the service provider system 110 to track order fulfillment. For example, the encounter management system 104 may establish an order by communicating order data that includes a tracking number or order identifier to the accounting system 118. In response to receiving order data 138 or in response to receiving order results data 128, a service provider system 110 may communicate with the accounting system 118 to indicate a further status of the order. In addition, the service provider system 110, after optionally communicating with a consultant system 114 and receiving consultant input data 130, may communicate with the accounting system 118 to indicate the status of an order. Further, the service provider system 110 or the encounter management system 104 may communicate with the accounting system 118 to indicate completion of an order in response to completion of an order. The accounting system 118 may store the accounting data 134 associated with orders and their status in a memory 132. In an exemplary embodiment, the accounting system 118 may pay fees to a third party facilitator based at least in part on the accounting data 134. In addition, the accounting system 118 may store a list 136 of service providers. For example, the list 136 may include information about service providers that have an agreement to provide services through a fulfillment network.

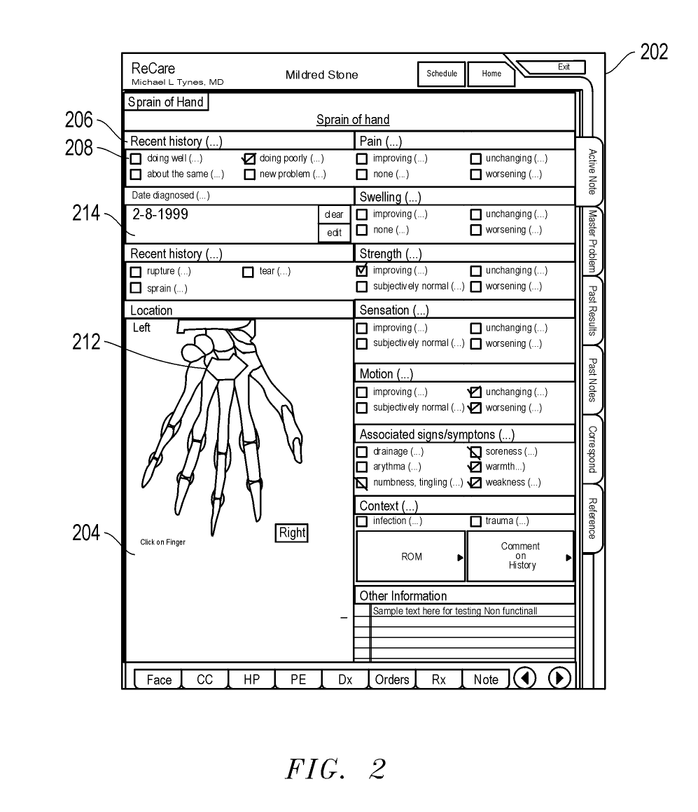

FIG. 2 includes an illustration of an exemplary interface for receiving discrete medical findings. In an exemplary embodiment, the encounter management system 104 provides such an interface to an input device accessible by a healthcare professional. The exemplary interface 202 includes controls for entering the discrete findings. An exemplary control includes a tri-state control, a bi-state control, a pop-up window, a radio button, a button, a drop-down menu, a text entry control, a handwriting recognition control, or any combination thereof. For example, the control 206 represents a heading that may be selected for entry of additional information. In addition, control 206 may also include sub-headings 208, which may be selected, crossed through, or left blank to indicate positive, negative, or undetermined associations with the sub-heading. As illustrated, the control 206 is implemented as a checkbox. In another example, the control may be implemented as a binary control or three-way control. For example, under a recent history heading 206, a healthcare professional may select a sub-heading “doing poorly” while leaving others blank. In another example, a text box control 214 may be implemented to allow entry of text, such as through handwriting recognition. In a further example, graphical images, such as image 204 may be provided that have selectable areas, such as area 212 for indicating locations of findings.

Data entered on the interface 202 may be communicated with the encounter management system, which may store the findings data in a database. In a particular embodiment, the encounter management system stores the findings as discrete codes associated with the patient record, each code indicating a specific finding. Alternatively, the finding may be stored in accordance with a medical vocabulary such that key words indicate the findings. In general, a finding is a single data element indicating a condition, a diagnosis, a complaint, a quality associated with a condition, a pathology, or any combination thereof. In particular, a discrete medical finding indicates a code or word of a medical vocabulary indicative of a finding. In an exemplary embodiment, the discrete medical finding is associated with a symptom. In another example, discrete medical finding is associated with a chief complaint. In a further example, the discrete medical finding is associated with a physical exam. In addition, the discrete medical finding may be associated with a diagnosis.

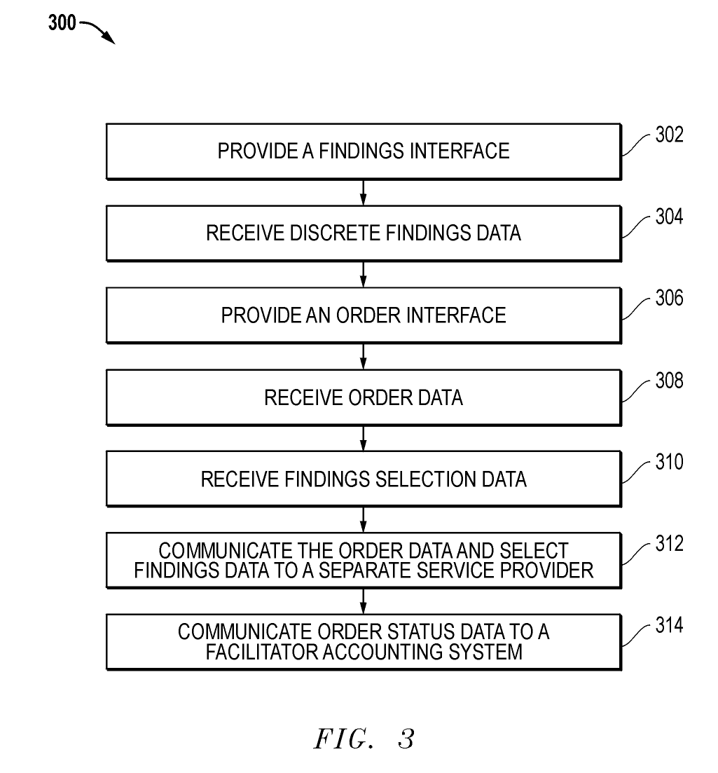

In an exemplary embodiment, the encounter management system communicates via an input device with a healthcare provider to acquire discrete medical findings and order data. The encounter management system provides a select portion of the discrete medical findings and the order data to a separate service provider for fulfillment of the order. As illustrated in FIG. 3, a method 300 for facilitating an order includes providing a findings interface to an interface device, as illustrated at 302. As a result, the encounter management system may receive discrete findings data, as illustrated at 304. Within the medical workflow, the encounter management system 104 may also provide an order interface, as illustrated at 306, and in response, receive order data indicative of a desired order, as illustrated at 308. In an exemplary embodiment, the order data may be indicative of an order to be fulfilled at a separate service provider location. For example, the order may be a laboratory order to be performed at a laboratory site. In another example, the order may be a radiological order, such as an order for an X-ray, CAT scan, or MRI.

In an embodiment, a patient may be directed to the service provider to facilitate completion of the order. For example, in the case of a laboratory, a patient may be directed to a laboratory location for bodily fluid sampling, such as urine sampling or blood sampling. In another example, a patient may be directed to an imaging facility for performing an X-ray, a CAT scan, or an MRI.

In addition to receiving the order data, the encounter management system 104 may receive findings selection data, as illustrated at 3 10. In an exemplary embodiment, the orders interface may include an interface for selecting a particular portion of the findings or the findings as a whole to accompany the order. In another exemplary embodiment, the findings selection data may be entered in a separate interface from the orders interface. Alternatively, the findings selection data may be stored as a default within the encounter management system 104. The findings selection data indicates particular findings that may accompany the order. For example, the findings selection data may indicate that current discrete medical findings associated with the current patient visit may be attached to the order data forwarded to a service provider. In another example, the findings selection data may indicate that all of the patient's medical history is to be included with or attached to the communication of the order data. In a further example, a healthcare provider may select a subset of the discrete medical findings associated with the patient to be included with the order data. The order data may, for example, include an order identifier and specific information about the order, such as which tests are to be performed, which methodologies are to be followed, and contact information for the patient. For example, a laboratory order may include an order identifier, such as an identification number, patient information, such as a contact number or insurance information, and specific data associated with the order, such as, for an exemplary blood test, a request for performing a blood glucose level, an HIAC test, a total cholesterol test, and a triglyceride test. For an exemplary radiological order, the order data may include an order identifier, patient contact information, the type of radiological test, such as X-ray, CAT scan, or MRI, and an anatomical location. In a particular embodiment, radiological order data may include an anatomical drawing of a body or a portion of the body with an indicator of the location of which the healthcare provider desires imaging.

On receiving the order data and the findings selection data, the encounter management system may communicate the order data and select findings data to a separate service provider located at a separate and distinct ambulatory location, as illustrated at 312. The select findings data may, for example, be a set of discrete medical findings that are selected by the healthcare provider and indicated by the findings selection data received from an input device via an interface. For example, the encounter management system may communicate with a separate service provider system located at a remote location. In addition, the encounter management system communicates an order status data to a facilitator accounting system, as illustrated at 314. The facilitator accounting system 314 may establish an order within the facilitator accounting system 314 and await further order status from a service provider system.

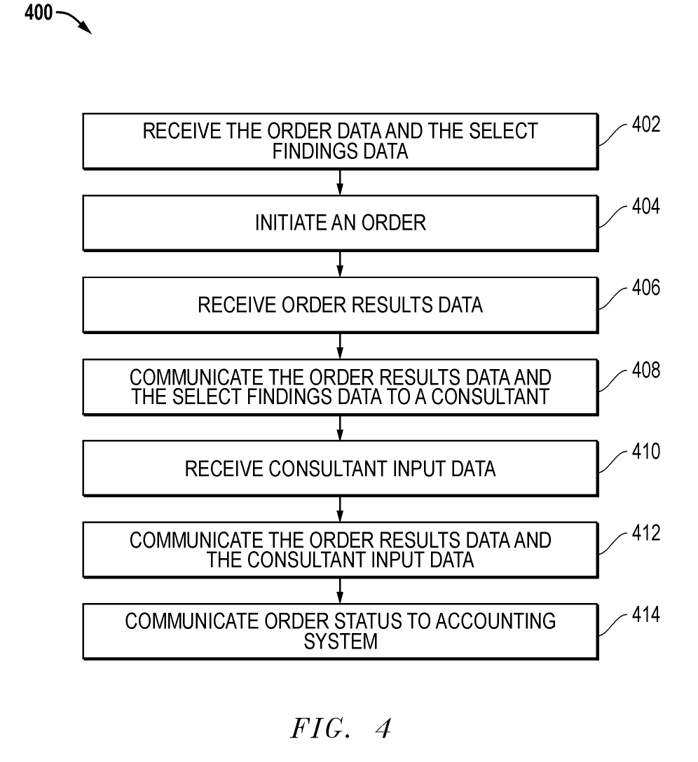

As illustrated in method 400 of FIG. 4, the service provider system receives the order data and the select findings data, as illustrated at 402. In response, the service provider system initiates the order, as illustrated at 404. Initiation of the order may include contacting a patient and establishing a schedule. In another exemplary embodiment, initiating an order may include establishing a record into which order results may be entered. For example, in a laboratory setting, a system may initiate an order by establishing an order record. Once samples have been acquired, and the tests have been performed, the order results may be entered into the record. In a radiological example, an order initiation may include establishing an order record, prompting contact with the patient to establish a schedule or appointment, and facilitate order taking, such as requesting preauthorization from a third party payer, such as an insurance company or a government entity.

Once the ordered tests have been performed, the service provider system 104 may receive the order results data, as illustrated at 406. For example, the order results data may include numerical test results of the blood test. In another example, the order data may include radiological images, such as X-ray images, MRI images, or CAT scan images. In addition, the order results data may include an order identifier, such as an order number or may include a clinic identifier, such as a clinic number associated with a requesting clinic.

Optionally, the service provider system may communicate the order results data and the select findings data to a consultant system, as illustrated at 408. For example, a radiologist may have a separate computer system that receives radiological images for analysis. The consultant may enter findings associated with the review or analysis of the order results data. For example, a radiologist may enter findings associated with analysis of an X-ray image. In a particular embodiment, the findings may be entered as discrete medical findings. In another embodiment, the findings may be entered as text, an audio dictation, a visual indication on an image associated with a test, a multimedia element, or any combination thereof. The consultant input data may be forwarded to the service provider system. Alternatively, the consultant input data may be forwarded to the encounter management system directly.

A service provider system receives the consultant input data, as illustrated at 410. In response, the service provider system communicates the order results data and optionally, the consultant input data to the encounter management system, as illustrated at 412. In an exemplary embodiment, the service provider system communicates the order results data and the consultant input data together within the same communication. Alternatively, the service provider system may communicate the order results data and optionally, the consultant input data separately in different communications. For example, the service provider system may communicate the order results data when the order results data is received and may communicate the optional consultant input data when it is received.

In addition, the service provider system communicates the order status to the accounting system, as illustrated at 414. For example, the service provider system may indicate to the accounting system that the order is complete. The accounting system may update the status of the order within a database.

In the case of radiological data, the order results data is frequently acquired in a format, such as digital imaging and communications in medicine (DCIM) format. As such, the service provider system may convert the radiological image into a different format, as illustrated in method 500 of FIG. 5. For example, the service provider system may receive DCIM-formatted data as part of the order results data, as illustrated in 502. The service provider system may convert the DCIM format data to compressed Internet format data, such as JPEG, GIF, MPEG, Bitmap, PNG, or any combination thereof. The compressed Internet format data may be included in the order results data or substituted for the DCIM format data in the order results data.

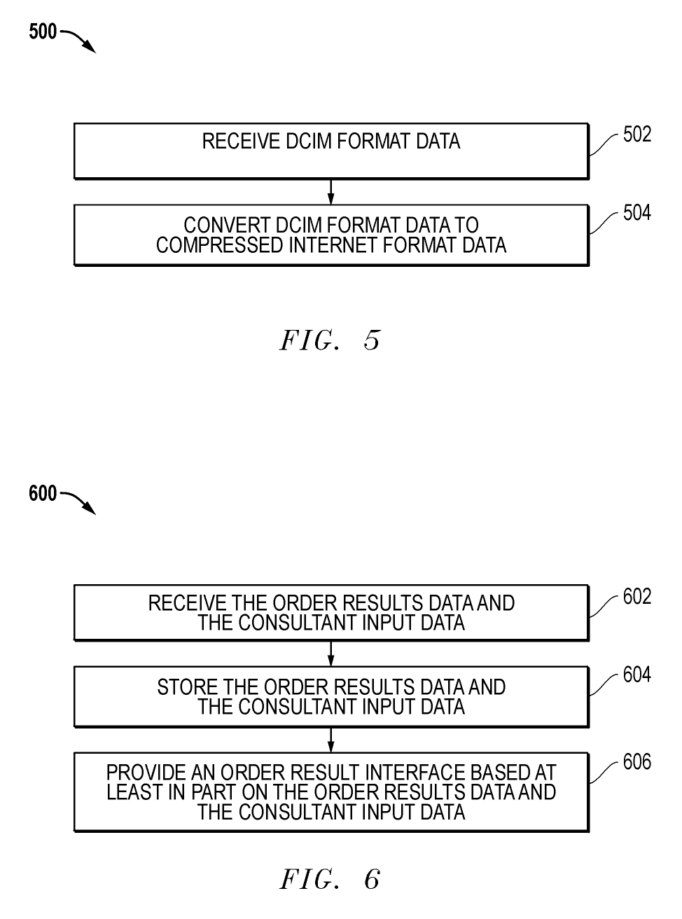

When communicated from the service provider system, the encounter management system receives the order results data and optionally the consultant input data, as illustrated at 602 of method 600 of FIG. 6. The encounter management system stores the order results data and the optional consulting input data, as illustrated at 604. In an exemplary embodiment, the encounter management system stores the order results data in a discrete medical findings database. In a particular example, images are stored on the encounter management system and an indicator is stored within the encounter management system's discrete findings database indicating the presence of the image. When consultant data is included in a discrete medical findings format, the discrete medical findings data provided in the consultant input data may be stored directly into the discrete medical findings database of the encounter management system. Alternatively, the consultant input data may include a text-based statement, which may be incorporated into a text file or field within a database.

When accessed by a healthcare professional, the encounter management system may provide an order results interface based at least in part on the order result data and optionally, the consultant input data, as illustrated at 606. For example, an interface may be developed from the discrete medical findings provided by the order results data and optionally, the consultant input data. In another exemplary embodiment, an interface may be provided that includes radiological image data or text provided within the consulted input data.

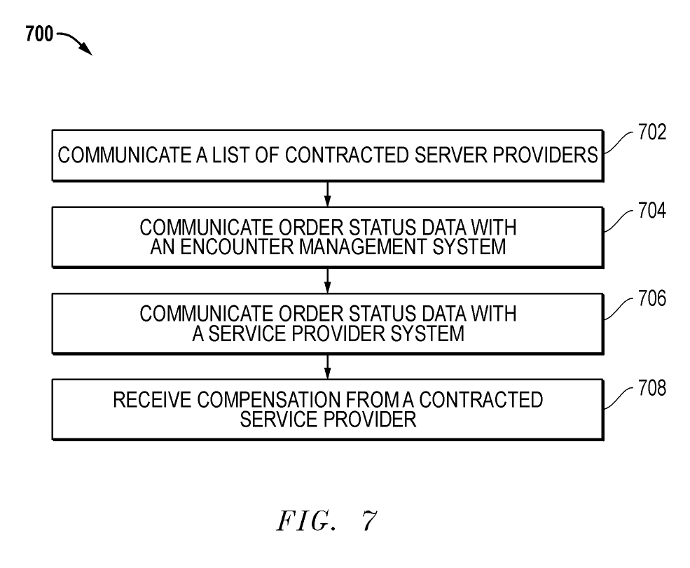

The process for facilitating order fulfillment may be implemented by a third party provider. In an exemplary method 700 illustrated in FIG. 7, the third party provider's accounting system interacts with the encounter management system or service provider system. For example, the facilitator may communicate a list of contracted service providers to the encounter management system, as illustrated at 702. When placing an order, the encounter management system may provide an interface to a healthcare provider for selecting a service provider from the list of contracted service providers. Once an order is placed, the encounter management system may communicate order status data with an accounting system of the facilitator, as illustrated at 704. The order status data may be communicated after placing an order with the service provider system, before placing an order with the service provider system, or may include the order data so that the facilitator accounting system may communicate that data to the service provider system. The service provider system communicates order status data with the facilitator accounting system, as illustrated at 706. For example, the service provider system may provide the accounting system updates as to the order status. In particular, the service provider system may indicate initiation of an order to the accounting system. In another example, the service provider system may indicate completion of the order and may provide the order results data to the accounting system.

In a particular embodiment, the third party facilitator receives compensation from a contracted service provider, as illustrated at 708. In an exemplary embodiment, the compensation is based at least in part on the order facilitated by the third party facilitator. For example, the service provider may compensate the third party facilitator based on a number of orders placed and completed. In another example, the service provider may provide a flat fee to the facilitator based upon an order number parameter. In a further example, the service provider may be contracted using a combination of flat fee and volume based pricing.

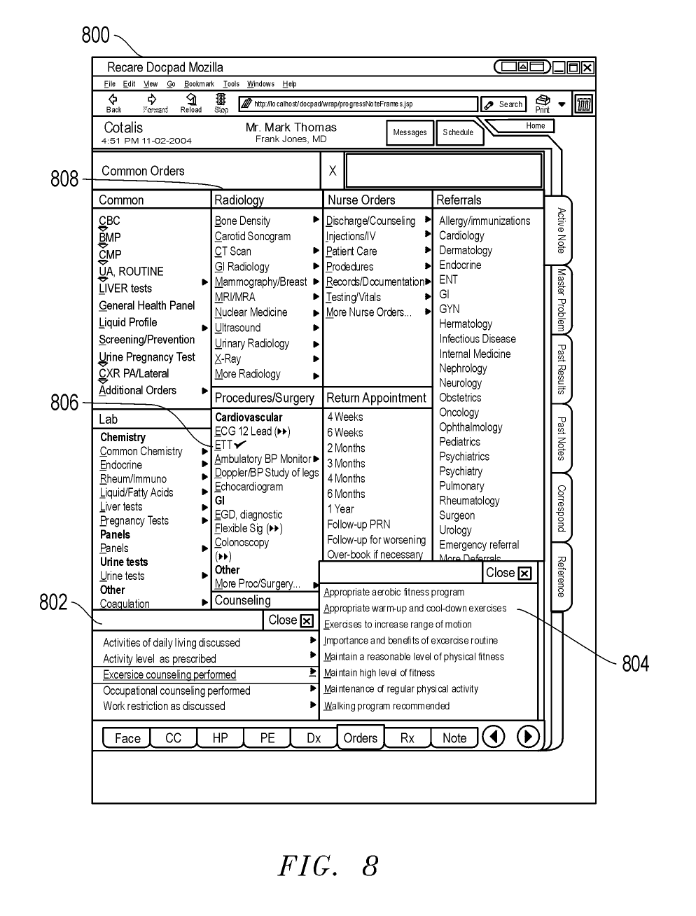

FIGS. 8, 9, 10, 11, 12, 13, and 14 include illustrations of exemplary interfaces for facilitating an order. For example, the encounter management system may provide such interfaces to an input device accessible by a healthcare provider. FIG. 8 includes an illustration of an interface 800 that includes controls to enter order parameters. Exemplary controls include tri-state controls, bi-state controls, pop-up windows, text controls, radio buttons, buttons, drop-down menus, text entry controls, and handwriting recognition controls. For example, a healthcare provider may select controls, as indicated by a background checkmark on the exemplary interface. As illustrated, control 806 includes a checkmark indicating a procedure ETT is to be performed.

Order types may be organized by headings. In a particular embodiment, a radiology heading 808 includes controls to select radiological tests, such as bone density tests, sonograms, CT scans, GI radiology test, mammographies, MRIs, nuclear medicine test, ultrasounds, urinary radiology tests, X-rays, or additional options. For those subcategories that include further subcategories, pop-up windows, such as 802 and 804 may be provided for navigating through the options.

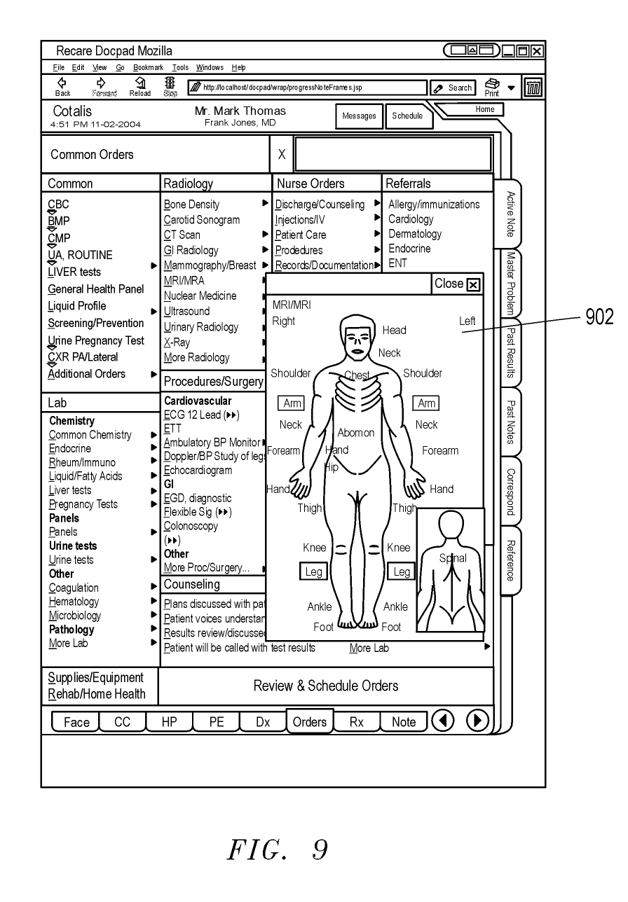

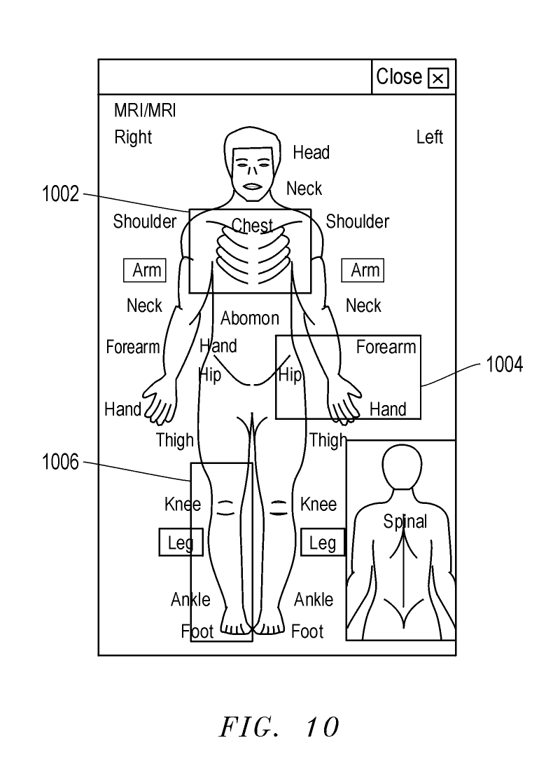

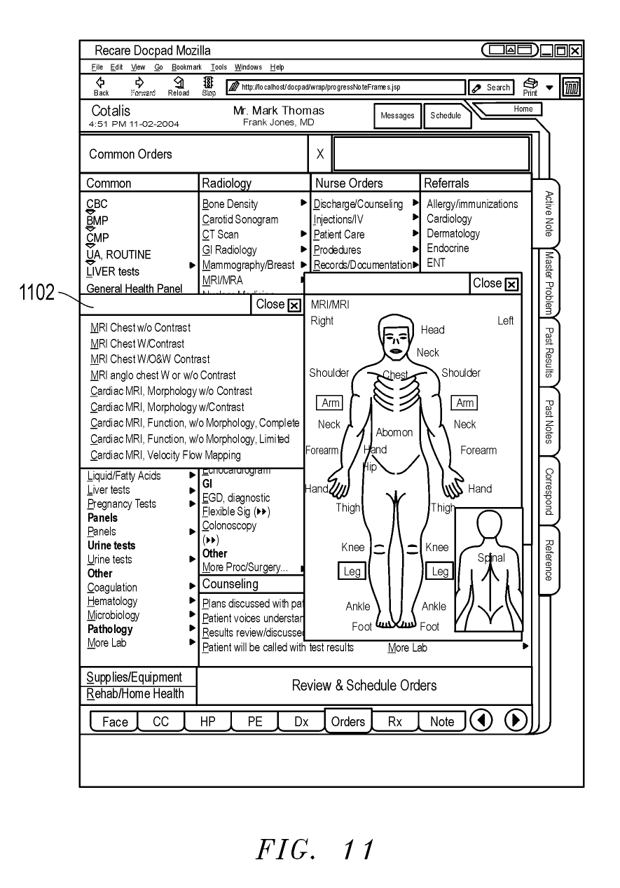

As illustrated in FIG. 9, a healthcare professional may select a radiology order, such as an MRI. As illustrated, a pop-up window 902 may permit entry of a location indicating an anatomical location of which a medical professional desires an image. As illustrated in FIG. 10, the interface may be implemented to permit the medical professional to select particular of labels indicative of locations. Alternatively, the interface may be implemented to allow annotation and selection, such as through the drawing of squares on an anatomical drawing, as illustrated at 1002, 1004, and 1006. In a particular embodiment, the anatomical drawing may change to indicate selection of a region of the body, such as by indicating an internal representation of the body, such as a skeleton. Alternatively, the healthcare provider may be provided with a pop-up window, including a list of locations, as illustrated at 1102 of FIG. 11.

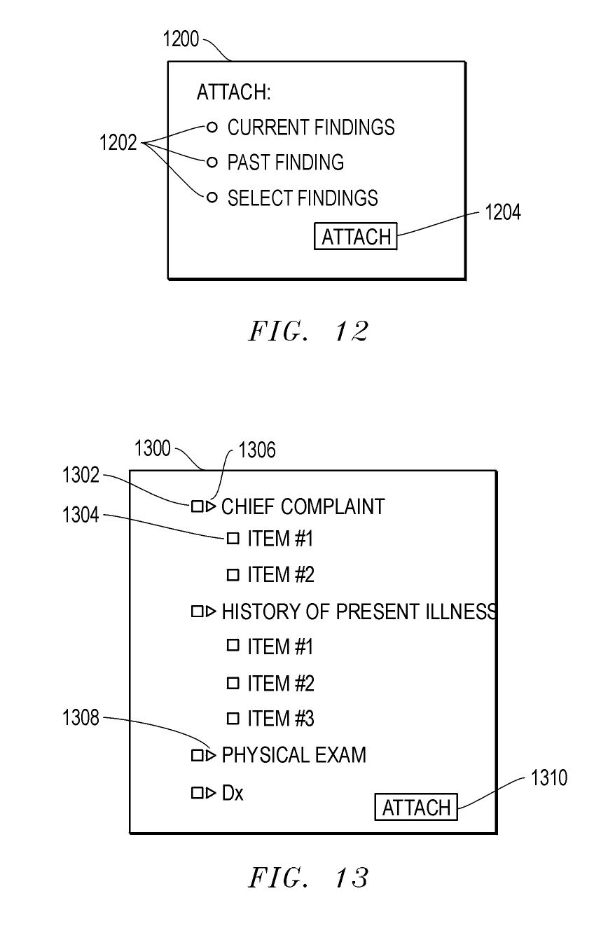

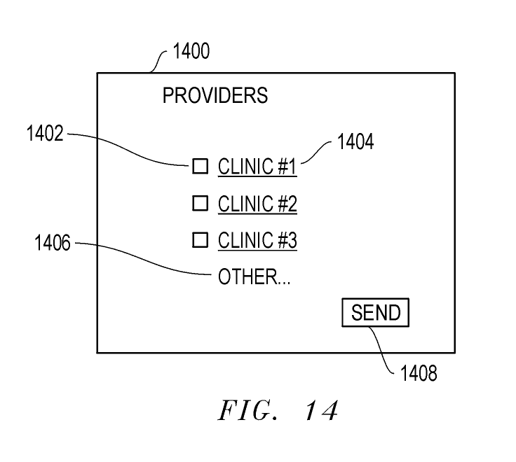

As part of the order interface or in a separate interface, the healthcare provider may be prompted to select findings data to be attached with or incorporated with the communication of the order data. FIGS. 12 and 13 include illustrations of exemplary interfaces for selecting findings. As illustrated in FIG. 12, an interface 1200 provides controls 1202 to select current findings, to select past findings, or to select to customize findings selection. In an embodiment, the interface 1200 may also include a control to allow selection of a defined set of findings or a defined level of disclosure. In addition, the interface 1200 may include a button 1204 to attach the selected findings to the order data. In response to selecting the customized findings option or as an alternative interface, an interface 1300 illustrated in FIG. 13 may be provided. The interface 1300 includes headings, such as chief complaint heading 1302, with control boxes that may be selected. In addition, the controls may include an indicator that permits selection of subheadings within a category, as illustrated at 1306 and 1308. For example, when the control is in a particular state, as indicated by 1306, additional controls under subheadings may be provided, such as controls 1304. Alternatively, the controls may be hidden, as indicated by the physical exam at 1308. Further, the controls may be populated based on a set of findings associated with a current patient encounter. Alternatively, the controls may be populated based on a set of stored findings, such as findings stored from past patient encounters. In addition, the interface 1300 may include a control to accept, to attach or to send the finding selection data to the encounter management system, as illustrated at 1310.

In an addition embodiment, to complete the order, the encounter management system may provide an interface 1400, as illustrated at FIG. 14. The interface 1400 includes a set of controls, such as control 1402, to select particular service providers. Data about these service providers may be included in the interface. Alternatively, hyperlinks to additional data about the service provider may be included as part of the label, as illustrated at 1404. In addition, a control to select additional or alternative clinics or service providers may be provided, as illustrated at 1406. Further, a control to send the order may be provided, as illustrated at 1408.

In a particular embodiment, a method of establishing an order includes receiving discrete medical findings data associated with a patient, receiving order data associated with the patient, receiving findings selection data indicative of at least a portion of the discrete medical findings data, and communicating the order data and select findings data to a separate service provider. The select findings data include at least the portion of the discrete medical findings data. In addition, a computer readable media may include computer-implemented instructions for performing such a method.

In another exemplary embodiment, method of performing a medical order includes receiving order data and select discrete medical findings data at a service provider system, receiving order results data associated with the order data at the service provider system, communicating the order results data and the select discrete medical findings data to a consultant system, receiving consultant input data from the consultant system at the service provider system, and communicating the order results data and the consultant input data to a encounter management system located at a remote ambulatory facility. In addition, a computer readable media may include computer-implemented instructions for performing such a method.

In a further exemplary embodiment, a method of performing a medical order includes receiving order data and select discrete medical findings data at a service provider system, receiving order results data associated with the order data at the service provider system, communicating the order results data to a encounter management system located at a remote ambulatory facility, and communicating an order status associated with the order data to a facilitator accounting system. In addition, a computer readable media may include computer-implemented instructions for performing such a method.

In an additional exemplary embodiment, a computational system includes an encounter management server located at an ambulatory medical facility, and a service provider system in communication with the encounter management server. The service provider system located at a separate and remote facility.

In a further exemplary embodiment, a method of generating a medical order includes providing an order interface to receive order data associated with a patient. The order interface includes a control to receive finding selection data. The method also includes communicating the order data and select findings to a service provider. The select findings associated with the finding selection data.

In an additional exemplary embodiment, a method of facilitating an order for a medical service includes providing a list of service providers to a medical encounter management system located at an ambulatory medical facility. The list of service providers is associated with order fulfillment for the medical service. The method also includes communicating accounting data with a service provider system located at a service provider location.

In a particular embodiment, a computational system includes a communication interface to an input device and storage. The storage includes computer implemented instructions operable to display an discrete findings entry interface, computer implemented instructions operable to display an order interface configured for entry of order data, and computer implemented instructions operable to display a select findings interface configured for selection of findings data to be included with the order data. In an embodiment, the select findings interface is configured to permit selection of a level of findings. In another embodiment, the select findings interface is configured to permit selection of a finding from a set of entered findings. The set of entered findings may be associated with a current patient visit. Alternatively, the set of entered findings may be associated with stored findings. Further, the computational system may include computer-implemented instructions operable to display a list of service providers associated with the order data. In an additional embodiment, the computational system may include a communication interface to a service provider system. The computational system may be configured to communicate the order data and findings data to the service provider system. Also, the computational system may include a communication interface to an accounting system. The system may be configured to communicate an order initiation to the accounting system based at least in part on the order data.

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. A method of establishing an order, the method comprising:
receiving discrete medical findings data associated with a patient;
receiving order data associated with the patient;
receiving findings selection data indicative of at least a portion of the discrete medical findings data; and
communicating the order data and select findings data to a separate service provider, the select findings data including at least the portion of the discrete medical findings data.

2. The method of claim 1, wherein the order data is associated with a radiological order.

3. The method of claim 1, wherein the order data includes anatomical graphical data.

4. The method of claim 3, wherein the anatomical graphical data includes an indicator of anatomical location.

5. The method of claim 1, wherein the at least a portion of the discrete medical findings data includes a discrete medical finding associated with a symptom.

6. The method of claim 1, wherein the at least a portion of the discrete medical findings data includes a discrete medical finding associated with a chief complaint.

7. The method of claim 1, wherein the at least a portion of the discrete medical findings data includes a discrete medical finding associated with a physical exam.

8. The method of claim 1, wherein the at least a portion of the discrete medical findings data includes a discrete medical finding associated with a diagnosis.

9. The method of claim 1, wherein the discrete medical findings data include an indicia of a medical finding.

10. The method of claim 9, wherein the indicia includes a numerical code associated with the medical finding.

11. The method of claim 9, wherein the indicia includes a medical vocabulary tag associated with the medical finding.

12. The method of claim 1, further comprising providing a findings interface.

13. The method of claim 1, further comprising providing an order interface.

14. The method of claim 1, wherein the order interface includes a control to select at least a portion of the discrete medical findings data.

15. A system comprising:
a communication interface to an input device; and
storage comprising:
computer implemented instructions operable to display an discrete findings entry interface;
computer implemented instructions operable to display an order interface configured for entry of order data; and
computer implemented instructions operable to display a select findings interface configured for selection of findings data to be included with the order data.

16. The system of claim 15, wherein the select findings interface is configured to permit selection of a level of findings.

17. The system of claim 15, wherein the select findings interface is configured to permit selection of a finding from a set of entered findings.

18. (canceled)

19. (canceled)

20. (canceled)

21. (canceled)

22. (canceled)

23. A method of performing a medical order, the method comprising:
receiving order data and select discrete medical findings data at a service provider system;
receiving order results data associated with the order data at the service provider system;
communicating the order results data and the select discrete medical findings data to a consultant system;
receiving consultant input data from the consultant system at the service provider system; and
communicating the order results data and the consultant input data to a encounter management system located at a remote ambulatory facility.

24. The method of claim 23, further comprising communicating an order status associated with the order data to a facilitator accounting system located at a remote facility.

25. The method of claim 23, further comprising converting the order results data from a first format to a second format.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
